(19) United States
(12) Patent Application Publication
(10) Pub. No.: US 2025/0235405 A1
(43) Pub. Date: Jul. 24, 2025

(54) A COMPOSITION COMPRISING A THERAPEUTICALLY ACTIVE AGENT PACKAGED WITHIN A DRUG DELIVERY VEHICLE

(71) Applicant: BRANCA BUNUS LIMITED, Dublin (IE)

(72) Inventors: Wenxin Wang, Dublin (IE); Zhonglei He, Dublin (IE); Sigen A., Dublin (IE); Yinghao Li, Dublin (IE); Irene Lara-Saez, Dublin (IE); Xianqing Wang, Dublin (IE); Hongyun Tai, Dublin (IE)

(21) Appl. No.: 18/711,520

(22) PCT Filed: Nov. 21, 2022
(86) PCT No.: PCT/EP2022/082651

§ 371 Date: May 17, 2024

(30) Foreign Application Priority Data
Nov. 19, 2021 (EP) 21209432.0

(51) Int. Cl. A61K 9/14; A61K 9/19; A61K 31/7088

(52) U.S. Cl. CPC A61K 9/145; A61K 9/19; A61K 31/7088

(57) ABSTRACT

A nanoparticulate composition comprising a core comprising a therapeutically active agent selected from a nucleic acid, protein or peptide packaged within a non-viral drug delivery vehicle is described. The core comprises a low molecular weight stabilizing agent comprising at least one atom or group that is charged in aqueous solution, wherein when the therapeutic agent is negatively charged the charged atom or group is positively charged in aqueous solution and when the therapeutic agent is positively charged the charged atom or group is negatively charged in aqueous solution.

### FIELD OF THE INVENTION

The present invention relates to a composition comprising a therapeutically active agent packaged within a drug delivery vehicle. Also contemplated are methods of making the composition and the use of the composition to treat a subject.

### BACKGROUND TO THE INVENTION

Many delivery platforms, including polymer-based, viral-based, lipid-based, protein/peptide-based and other vectors, have been developed to deliver nucleic acids, including DNA and RNA. However, the current commercial RNA delivery techniques based on lipid nanoparticles for the COVID vaccine lack RNA stability and are vulnerable to high temperatures. The cholesterols formulated in COVID vaccines support LNP structure and provide the hydrophobic condition, which may help prevent mRNA hydrolysis, but showed no significant effect.

None of these RNA delivery vectors have a specific component designed to stabilise RNA. Small interfering RNA (siRNA) and short single-stranded RNA is much more stable than long single-stranded RNA, which results in the different RNA types demonstrating different physiochemical properties Therefore, the technologies for siRNA cannot be directly adapted for the delivery of long RNA or other more delicate bioactive molecules.

On the other hand, RNA stabilising techniques have been developed for a long time for in vitro examination. Freezing at ultralow temperature and specific solutions (i.e., ethylenediaminetetraacetic acid (EDTA), tris or sodium citrate) can stabilise/protect RNA by slowing down hydrolysis, limiting deprotonation of the 2′ hydroxyl group or inactivating metal ion-requiring enzyme (i.e., RNase). However, those techniques are not sensitive enough and only stabilise RNA at a limited level. They are also not cost-effective, practical during the administration route, or designed for RNA encapsulation within delivery vectors and may affect many downstream applications.

Specifically, RNA-protecting methods and reagents previously disclosed in patents, such as U.S. Pat. No. 6,204,375B1 & U.S. Pat. No. 6,528,641B2, WO2014146780A1, WO2017162518A1 and EP2765203A1, are more related to the field of molecular biology, known and described for RNA preservation in tissue samples, inhibition of RNA-cleaving/damaging molecules in RNA containing samples/mixture, or RNA stabilisation under high temperature and alkaline condition. None of these have been invented or applied to the formulation of RNA into delivery vectors for transfection and clinical application. None of them use the specific SMART structures found by the inventor.

Furthermore, reagents involved in those existing RNA-protecting patents, for example (1) EDTA, mentioned in many of those invented methods, is known to affect downstream applications; (2) sodium citrate, used as a standard buffer for lipid nanoparticle-RNA formulation, but has not demonstrated an improvement of the ¹efficacy of RNA transfection.

For some virus-based and protein-based delivery systems that encapsulate RNA within cells, the cell-based encapsulation and manufacturing system needs to be customised for different RNA cargos. This, combined with downstream purification and quality control processes, leads to significantly high costs and potential risks. Thus, if any, extracellular manufacturing of virus/protein-based delivery systems may require the addition of RNA protecting components.

The current techniques for DNA gene-editing associated systems and other large bioactive molecules also suffer from similar stability issues at different levels and have no specific component in the delivery systems.

In recent year, many efforts have been made to develop nucleic acid delivery vehicles that can withstand metabolic breakdown, while also penetrating cell membranes, in order to enhance the efficiency and efficacy of nucleic acid drug delivery to the target. One such example of this is the development of a polysaccharide-mediated nucleic acid delivery vehicle (WO2009036022A1). This strategy exploits polysaccharides (for example, chitosan) to introduce secondary and tertiary amines into the polymer structure through small molecule conjugation thereby increasing solubility, enhancing buffering capacity and endosomal escape, and facilitating cytoplasmic release of the complexed nucleic acid.

Another strategy employed to enhance nucleic acid delivery is the use of lipid nanoparticles (WO2012170930A1). Encapsulating the nucleic acid into these amine-containing lipid nanoparticles can both facilitate improved cellular uptake of the nucleic acid as well as endosomal escape of the nucleic acid into the cytoplasm This strategy has been further modified to incorporate cationic and/or ionizable amino lipids, and phospholipids including polyunsaturated lipids, PEG lipids, and structural lipids in specific fractions (WO2016118724A1). Manipulation of the fractions of lipids used can result in enhanced nucleic acid delivery to its target.

CRISPR-Cas 9 systems have emerged as a powerful technology that facilitates the targeting and subsequent engineering of nucleic acids. Recently, efforts have been made to improve the delivery of these systems to their target with the use of particle delivery components (WO2015089419A2). This invention provides methods for using elements of the CRISPR-Cas system by a particle delivery formulation as a means to modify a target polynucleotide. The particles of the delivery formulation include liposomes, nanoparticles, exosomes, and microvesicles.

It is an object of the invention to overcome at least one of the above-referenced problems.

### SUMMARY OF THE INVENTION

The Applicant has discovered that admixing a bioactive agent such as a nucleic acid with a low molecular weight stabilising agent that carries at least one atom or group that is positively charged in aqueous solution can improve the transfection efficiency of the nucleic acid when it is packaged in a suitable drug delivery vehicle such as a polymeric, lipidic or virus-based vector. The use of a stabilising agent has also been found to increase the stability of the nucleic acid by reversible binding to auto-hydrolysis susceptible sites in nucleic acid while not affect encapsulation efficiency within delivery vectors. Furthermore, these unexpected small molecule admixtures were found to contribute to the stability and efficacy of the delivery of other nucleic acids and gene editing systems. For stabilisation of nucleic acids, the stabilising agent is generally a low molecular weight compound (e.g., less than 400 D), and carries at least one nitrogen or amine group that is charged in aqueous solution. Optimal stabilising agent include a primary amine, a short hydrocarbon or amine backbone, and a second amine which may be a primary, secondary or tertiary amine, or a heterocyclic group in which one ring atom is a nitrogen.

In a first aspect, the invention provides a composition comprising (a) a core comprising a therapeutically active agent selected from a nucleic acid, protein or peptide packaged within (b) a polymeric, lipidic or protein based drug delivery vehicle, characterized in that the core comprises a low molecular weight stabilizing agent comprising at least one atom or group that is charged in aqueous solution, wherein when the therapeutic agent is negatively charged the charged atom or group is positively charged in aqueous solution and when the therapeutic agent is positively charged the charged atom or group is negatively charged in aqueous solution.

The invention also provides a composition formed by
(a) admixing a therapeutically active agent selected from a nucleic acid, protein or peptide with a low molecular weight stabilizing agent comprising at least one atom or group that is charged in aqueous solution, wherein when the therapeutic agent is negatively charged the charged atom or group is positively charged in aqueous solution and when the therapeutic agent is positively charged the charged atom or group is negatively charged in aqueous solution to form a stabilized therapeutically active agent;
(b) admixing the stabilized therapeutically active agent with drug delivery vehicle forming polymer, lipid or protein to form the nanoparticulate composition; and
(c) optionally, lyophilizing the nanoparticulate composition.

In the method of the invention, when nucleic acid was formulated, the nucleic acid is mixed with low molecular weight stabilizing agent such that the (stabilising agent) Positive Charge Group(nucleic acid)Phosphodiester Bond ratio is 500 to 0.1, 100 to 1, or 20 to 1, or 5 to 15.

In one embodiment, the composition has a particulate form. In one embodiment, the particulate composition has a particle size of less than 2 μm, 1.5 μm, 1000 nm, for example 20-900 nm, 50-800 nm, 50-700 nm, 50-600 nm, 50-500 nm, 50-400 nm, 50-300 nm, 100-300 nm.

In any embodiment, the therapeutically active agent is a nucleic acid.

In any embodiment, the low molecular weight stabilizing agent comprises a primary, secondary or tertiary amine.

In any embodiment, the low molecular weight stabilizing agent comprises a terminal hydroxyl group.

In any embodiment, the low molecular weight stabilizing agent is linear.

In any embodiment, the low molecular weight stabilizing agent comprises a hydrocarbon backbone with terminal hydroxy group at each end. The hydrocarbon backbone may include one or more ether groups.

In any embodiment, the low molecular weight stabilizing agent comprises a hydrocarbon backbone (with, e.g., 2 to 10 carbon atoms) with a terminal hydroxy group at one end and an amine at an opposite end. The amine may be a primary, secondary or tertiary amine.

The hydrocarbon backbone may include one or more ether groups.

In any embodiment, the low molecular weight stabilizing agent is a diamine.

In any embodiment, the low molecular weight stabilizing agent has a chemical formula R₁-L₁-R₂:
in which:
R₁ is selected from a primary, secondary or tertiary amine, or a hydroxyl group;
L₁ is a linker; and
R₂ is selected from a primary, secondary or tertiary amine, or a hydroxyl group, or is absent.

In any embodiment, L₁ is an alkyl group which is straight or branched and optionally substituted.

In any embodiment:
R₁ is NH₂;
L₁ is selected from a straight or branched amine or alkyl group having 2 to 8 carbon atoms; and
R₂ is selected from a primary, secondary or tertiary amine.

In any embodiment:
R₁ is OH;
L₁ is selected from a straight or branched amine or alkyl group having 2 to 8 carbon atoms, and optionally one or two oxygen atoms; and
R₂ is selected from OH or is absent.

In any embodiment, R₂ is selected from the groups consisting of: NH₂; N(CH₃)₂; and a heterocyclic group.

In any embodiment, R₂ is N(CH₃)ₙOH, in which n=1 to 5.

In any embodiment, the heterocyclic group contains one or more (e.g., 2 or 3) ring atoms each individually selected from nitrogen, oxygen, sulphur, phosphorous, and a halogen.

In any embodiment, the heterocyclic group contains a ring atom selected from nitrogen and oxygen.

In any embodiment, the heterocyclic group includes at least two heteroatoms.

In any embodiment, the at least two heteroatoms are nitrogen.

In any embodiment, the heterocyclic group contain one nitrogen heteroatom and one oxygen heteroatom.

In any embodiment, R₂ is a 4-membered or 5-membered heterocyclic group.

In any embodiment, R1 or R2 has a chemical structure:

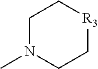

in which R3 is CH or comprises a heteroatom such as N or O.

In any embodiment, R3 is selected from CH, NH, O, N(CH₂)ₙCH₃, in which n is a whole number from 0 to 3.

In any embodiment, R3 is selected from:

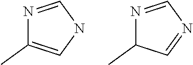

In any embodiment, L2 does not contain an ether group.

In any embodiment, the low molecular weight stabilizing agent is selected from:

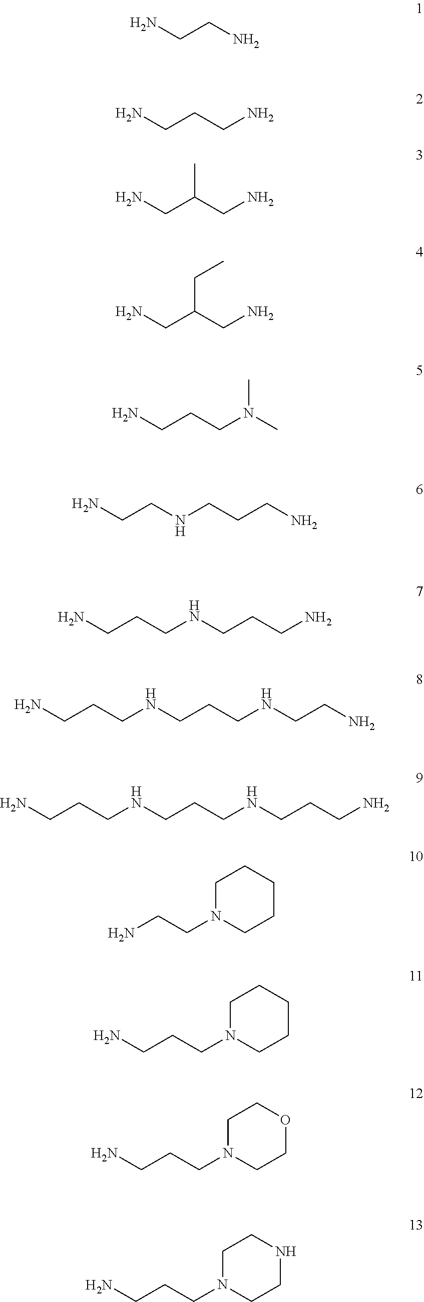

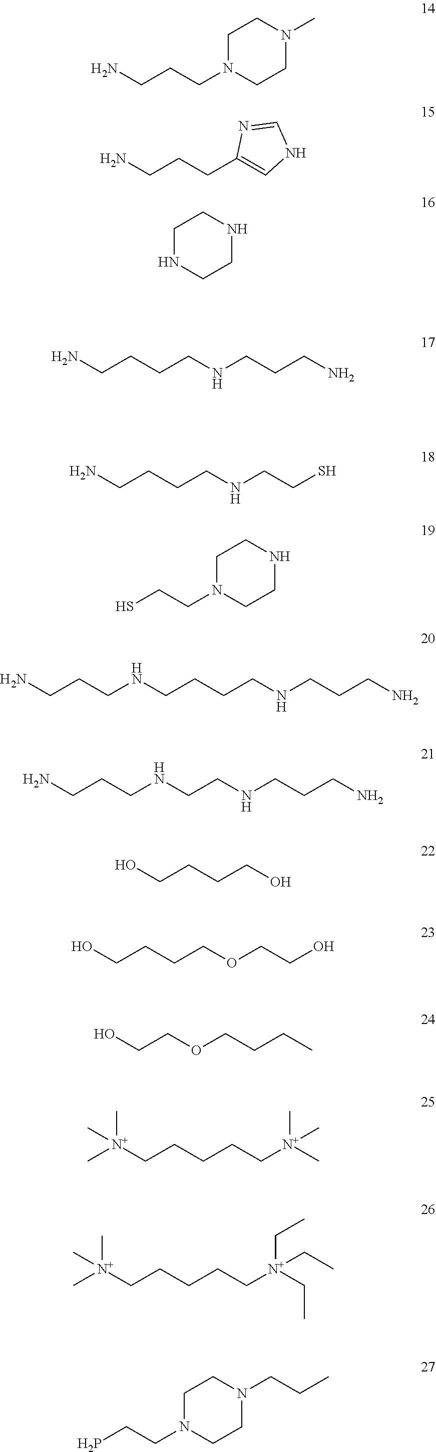

In any embodiment, the low molecular weight stabilizing agent has a molecular weight of less than 2000 Da, 1500 Da, 1000 Da, 500 Da, 400 Da, 300 Da, 250 Da or 200 Da.

In any embodiment, the therapeutically active agent is RNA.

In any embodiment, the therapeutically active agent is long RNA.

In any embodiment:
the therapeutically active agent is RNA;
the protective coating is a polymer, lipid or viral based coating; and
the low molecular weight stabilizing agent has a chemical formula R₁-L₁-R₂:
in which:
R₁ is selected from a primary, secondary or tertiary amine;
L₁ is an alkyl group which is straight or branched and optionally substituted
R₂ is selected from a primary, secondary or tertiary amine.

In any embodiment:
the therapeutically active agent is RNA.
the therapeutically active agent is long RNA;
the protective coating is a cationic polymer; and
the low molecular weight stabilizing agent has a chemical formula R₁-L₁-R₂:
in which:
R₁ is selected from a primary, secondary or tertiary amine;
L₁ is an alkyl group which is straight or branched and optionally substituted
R₂ is selected from a primary, secondary or tertiary amine.

In any embodiment, the composition is provided in a lyophilized form.

In any embodiment, the stabilising agent contains less than 5, 4 or 3 charged atoms or groups.

The invention also provides a pharmaceutical composition comprising a composition of the invention in combination with a suitable pharmaceutical excipient.

The compositions of the invention may be employed in therapy, e.g., gene therapy and in particular in gene addition, gene replacement, gene knockdown and gene editing. Gene replacement is defined as the provision of a functional healthy copy of a gene to replace a dysfunctional mutant containing gene which has given rise to a disease. Gene addition is defined as the supplementation of therapeutic genes that target a specific aspect of a disease mechanism. Gene knockdown is defined as the process of inhibiting a target genes capability to synthesize a toxic/dysfunctional protein which gives rise to a disease. Gene editing is defined as the process whereby a target genes nucleotide sequence is altered resulting in a loss of function/correction/manipulation of gene expression. Such gene editing systems consists of but are not limited to i) clustered, regularly interspaced, palindromic repeats (CRISPR)-associated (Cas) system; (ii) a transcription activator-like effector nuclease (TALEN) system; or (iii) a zinc finger nuclease (ZFN) system.

Other applications include
1). Vector for research use
2.) If combined with a probe the invention can be used as a cell/tissue marker for research or diagnostic
3.) If combined with a targeting moiety can be used to target specific cell types
4.) If combined with a pharmaceutical agent and a targeting moiety can be used to deliver a drug in a specific organ, tissue or cell type.

The invention also provides a method of treating a subject comprising administering a composition of the invention to the subject.

The invention also provides a method of transfecting a cell comprising a step of contacting one or more target cells with a composition of the invention under conditions suitable for transfecting the cell with the composition. Transfection may be in-vivo, ex-vivo, or in-vitro. Transfection may involve modification of the genome of the cell (for example by deleting all or part of the genome), inserting a sequence into the genome (insertational mutagenesis), silencing a gene, replacing a gene, upregulating expression of a gene, editing a genome (for example deleting disease causing mutations), and adding residues required for proper functioning of the gene.

Other aspects and preferred embodiments of the invention are defined and described in the other claims set out below.

### BRIEF DESCRIPTION OF THE FIGURES

FIG. 1 Scheme defining the concept of Small Molecules Admixture for Readily Transfection (SMART), using RNA as an example cargo.

FIG. 2 Luminescence 48-hour post-transfection of HEK293, screening an example library of SMART using HPAE-control.

FIG. 3 Luminescence 48-hour post-transfection of HEK293, five leading SMART candidates from example library, combined with Lipo MM, jetM and Xfect.

FIG. 4 Cell viability of HEK293 after 48 hours transfection with/without SMART.

FIG. 5 (a) mRNA entrapment and stability of SMART formulated and control groups; (b) Nanoparticle sizes of SMART formulated and control groups.

FIG. 6 Efficacy of liquid and lyophilised SMART-formulated mRNA-polyplex store at different temperatures.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g., the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g., the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g., the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

As used herein, the term “stabilising agent” refers to a low molecular weight moiety having one or more atoms or groups that are charged in aqueous solution. The stabilising agent is chosen such that when it is formulated with a therapeutically active agent that bears a negative charge (for example a nucleic acid) the stabilising agent bears a positive charge, and visa-versa. The stabilising agent may carry a positive charge in aqueous solutions via protonation, quaternisation, or similar reactions/processes. When formulated with a nucleic acid or another active agent that is negatively charged (e.g., as a result of phosphodiester bonds), the stabilising agent generally is a diamine, and usually comprises a primary amine and a further amine (such as a primary, secondary or tertiary amine or a nitrogen containing heterocyclic group. The stabilising agent may be linear or branched and may be substituted or unsubstituted. The stabilising agent may have a chemical formula R₁-L₁-R₂ in which R₁ is selected from a primary, secondary or tertiary amine, or a hydroxyl group, L₁ is a linker, and R₂ is selected from a primary, secondary or tertiary amine, or a hydroxyl group, or is absent. L₁ may be an alkyl group which is straight or branched and optionally substituted and may contain one or more ether groups.

As used herein, the term “low molecular weight” as applied to a stabilizing agent means having a molecular weight of less than 500 D. In one embodiment, the low molecular weight stabilizing agent has a molecular weight of less than 400 D, 300 D, 250 D or 200 D.

As used herein, the term “core” refers to the part of the nanoparticulate composition that is packaged within the non-viral drug delivery vehicle. It typically contains a therapeutically active agent such as a nucleic acid and the stabilizing agent.

The stabilized therapeutically active agent is packaged within a polymer, lipid, protein (or peptide) based or any other non-viral drug delivery vehicle. Examples of polymer based drug delivery vehicles include cationic polymers, poly-beta amino esters, and hyperbranched polymers (i.e. chitosan¹, DEAE-dextran², poly(L-lysine)³, polyethyleneimine (PEI)⁴ and many other block copolymers and derivatives). Examples of lipid based drug delivery vehicles include lipid nanoparticles formulated by Lipofectamine⁵, C12-200⁶, 306Oᵢ₁₀⁷, OF-02⁸, TT3⁹, 5A2-SC8¹⁰, SM-102 (Moderna vaccine)¹¹ and ALC-0315 (Pfizer-BioNTech vaccine)¹² together with or without cholesterol, helper lipids, PEG-lipids or other excipients. Examples of protein-based drug delivery vehicles include PepFect14¹³, protamine¹⁴ and virus-like protein PEG10¹⁵. Examples of other non-viral drug delivery vehicles include cationic nanoemulsions (i.e. squalene-based formulations^{16,17}).

“Therapeutically active agent” refers to a nucleic acid, protein or peptide, or any analogue/variant thereof (e.g., PNA, LNA), or other therapeutically active agent that is susceptible to spontaneous self-cleavage (auto-hydrolysis) reactions. The agent is typically a nucleic acid, for example DNA or RNA. In one embodiment, the RNA is a long RNA (also known as large RNA), for example messenger RNA (mRNA) and long non-coding RNA (lncRNA). DNA also is susceptible to hydrolysis under acidic conditions or with the presence of enzymes. The nucleic acid is generally single stranded.

“Linker” means any linker group, including a linear or branched, substituted or unsubstituted, aryl or alkyl group. Preferred linkers include alkyl, lower alkyl, alkoxy, lower alkoxy groups.

“Diamine” refers to a moiety having one functional NH₂ group connected to an amine group by a linker. The diamine generally includes a hydrocarbon backbone.

“Alkyl” refers to a group containing from 1 to 10 carbon atoms and may be straight chained or branched. An alkyl group is an optionally substituted straight, branched or cyclic saturated hydrocarbon group. When substituted, alkyl groups may be substituted with up to four substituent groups, at any available point of attachment. When the alkyl group is said to be substituted with an alkyl group, this is used interchangeably with “branched alkyl group”. Exemplary unsubstituted such groups include methyl, ethyl, propyl, isopropyl, a-butyl, isobutyl, pentyl, hexyl, isohexyl, 4, 4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), Haloalkyl (such as CC13 or CF3), alkoxy, alkylthio, hydroxyl, carboxy (—COOH), alkyloxycarbonyl (—C(O)R), alkylcarbonyloxy (—OCOR), amino (—NH2), carbamoyl (—NHCOOR— or —OCONHR), urea (—NHCONHR—) or thiol (—SH). Alkyl groups as defined may also comprise one or more carbon double bonds or one or more carbon to carbon triple bonds.

“Lower alkoxy” refers to O-alkyl groups, wherein alkyl is as defined hereinabove. The alkoxy group is bonded to the core compound through the oxygen bridge. The alkoxy group may be straight-chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

“Halogen” means the non-metal elements of Group 17 of the periodic table, namely bromine, chlorine, fluorine, iodine and astatine.

The terms “alkyl”, “cycloalkyl”, “heterocycloalkyl”, “cycloalkylalkyl”, “aryl”, “acyl”, “aromatic polycycle”, “heteroaryl”, “arylalkyl”, “heteroarylalkyl”, “amino acyl”, “non-aromatic polycycle”, “mixed aryl and non-aryl polycycle”, “polyheteroaryl”, “non-aromatic polyheterocyclic”, “mixed aryl and non-aryl polyheterocycles”, “amino”, and “sulphonyl” are defined in U.S. Pat. No. 6,552,065, Column 4, line 52 to Column 7, line 39.

“Halogen” means the non-metal elements of Group 17 of the periodic table, namely bromine, chlorine, fluorine, iodine and astatine.

### Gene Therapy/Editing

The present invention may be used to edit a portion of the genome of a cell or replace a portion of the genome of a cell with an exogenous DNA insert in an orientation-specific manner.

Thus, the invention may be used to edit or replace a defective portion of a disease-causing gene (e.g., for gene repair), or to insertionally inactivate (e.g., silence) a gene the expression of which is associated with a disease, or to edit or modify a gene for example to delete disease causing mutations or modify or add in residues required for normal functioning of a gene.

Thus, the invention finds application in gene therapy, as herein defined.

Gene therapies according to the invention may target all of the cells in an organism or may be targeted to a subset of cells (e.g., to selected organs, tissues or cells).

Gene therapies according to the invention may target somatic cells specifically.

Gene therapies according to the invention may exclude the targeting of germ line cells. It may exclude the targeting of totipotent cells. It may exclude the targeting of human embryos.

In cases where gene therapy according to the invention is applied to selected organs, tissues or cells, the method may be applied ex vivo to isolated organs, tissues or cells (e.g., to blood, blood cells, immune cells, bone marrow cells, skin cells, nervous tissue, muscle etc.).

Gene therapy finds application in the treatment of any genetically inherited disorder, particularly those arising from single gene mutations. Thus, gene therapy finds particular application in the treatment of lysosomal storage diseases, muscular dystrophies, cystic fibrosis, Marfan syndrome, sickle cell anaemia, dwarfism, phenylketonuria, neurofibromatosis, Huntington disease, osteogenesis imperfecta, thalassemia and hemochromatosis.

Other diseases which may be suitable for gene therapy according to the invention include diseases and disorders of blood, coagulation, heterogenous skin disease, cell proliferation and dysregulation, neoplasia (including cancer), inflammatory processes, immune system (including autoimmune diseases), metabolism, liver, kidney, musculoskeletal, neurological, neuronal and ocular tissues.

Exemplary skin diseases include recessive dystrophic epidermolysis bullose (RDEB), a rare heterogenous skin disease caused by biallelic loss-of-function mutations in the COL7A1 gene.

Exemplary blood and coagulation diseases and disorders include: anaemia, bare lymphocyte syndrome, bleeding disorders, deficiencies of factor H, factor H-like 1, factor V, factor VIII, factor VII, factor X, factor XI, factor XII, factor XIIIA, factor XIIIB, Fanconi anaemia, haemophagocytic lymphohistiocytosis, haemophilia A, haemophilia B, haemorrhagic disorder, leukocyte deficiency, sickle cell anaemia and thalassemia.

Examples of immune related diseases and disorders include: AIDS; autoimmune lymphoproliferative syndrome; combined immunodeficiency; HIV-1; HIV susceptibility or infection; immunodeficiency and severe combined immunodeficiency (SCIDs). Autoimmune diseases which can be treated according to the invention include Grave's disease, rheumatoid arthritis, Hashimoto's thyroiditis, vitiligo, type I (early onset) diabetes, pernicious anaemia, multiple sclerosis, glomerulonephritis, systemic lupus E (SLE, lupus) and Sjogren syndrome. Other autoimmune diseases include scleroderma, psoriasis, ankylosing spondylitis, myasthenia gravis, pemphigus, polymyositis, dermomyositis, uveitis, Guillain-Barre syndrome, Crohn's disease and ulcerative colitis (frequently referred to collectively as inflammatory bowel disease (IBD)).

Other exemplary diseases include: amyloid neuropathy; amyloidosis; cystic fibrosis; lysosomal storage diseases; hepatic adenoma; hepatic failure; neurologic disorders; hepatic lipase deficiency; hepatoblastoma, cancer or carcinoma; medullary cystic kidney disease; phenylketonuria; polycystic kidney; or hepatic disease.

Exemplary musculoskeletal diseases and disorders include: muscular dystrophy (e.g., Duchenne and Becker muscular dystrophies), osteoporosis and muscular atrophy.

Exemplary neurological and neuronal diseases and disorders include: ALS, Alzheimer's disease; autism; fragile X syndrome, Huntington's disease, Parkinson's disease, Schizophrenia, secretase related disorders, trinucleotide repeat disorders, Kennedy's disease, Friedrich's ataxia, Machado-Joseph's disease, spinocerebellar ataxia, myotonic dystrophy and dentatorubral pallidoluysian atrophy (DRPLA).

Exemplary ocular diseases include: age related macular degeneration, corneal clouding and dystrophy, cornea plana congenital, glaucoma, leber congenital amaurosis and macular dystrophy.

Gene therapy according to the invention finds particular application in the treatment of lysosomal storage disorders. Listed below are exemplary lysosomal storage disorders and the corresponding defective enzymes:
Pompe disease: Acid alpha-glucosidase
Gaucher disease: Acid beta-glucosidase or glucocerebrosidase
Fabry disease: alpha-Galactosidase A
GMI-gangliosidosis: Acid beta-galactosidase
Tay-Sachs disease: beta-Hexosaminidase A
Sandhoff disease: beta-Hexosaminidase B
Niemann-Pick disease: Acid sphingomyelinase
Krabbe disease: Galactocerebrosidase
Farber disease: Acid ceramidase
Metachromatic leukodystrophy: Arylsulfatase A
Hurler-Scheie disease: alpha-L-Iduronidase
Hunter disease: Iduronate-2-sulfatase
Sanfilippo disease A: Heparan N-sulfatase
Sanfilippo disease B: alpha-N-Acetylglucosaminidase
Sanfilippo disease C: Acetyl-CoA: alpha-glucosaminide N-acetyltransferase
Sanfilippo disease D: N-Acetylglucosamine-6-sulfate sulfatase
Morquio disease A: N-Acetylgalactosamine-6-sulfate sulfatase
Morquio disease B: Acid beta-galactosidase
Maroteaux-Lamy disease: Arylsulfatase B
Sly disease: beta-Glucuronidase
alpha-Mannosidosis: Acid alpha-mannosidase
beta-Mannosidosis: Acid beta-mannosidase
Fucosidosis: Acid alpha-L-fucosidase
Sialidosis: Sialidase
Schindler-Kanzaki disease: alpha-N-acetylgalactosaminidase

Gene therapies according to the invention also finds particular application in the treatment of proteostatic diseases including both aggregative and misfolding proteostatic diseases, for example prion diseases, various amyloidoses and neurodegenerative disorders (e.g., Parkinson's disease, Alzheimer's disease and Huntington's disease), certain forms of diabetes, emphysema, cancer and cystic fibrosis.

Gene therapies according to the invention finds particular application in the treatment of cystic fibrosis. Cystic fibrosis occurs when there is a mutation in the CFTR gene leading to reduced ion channel activity (via increased clearance of the misfolded CFTR proteins).

Gene therapies according to the invention finds particular application in the treatment of expanded CAG repeat diseases. These diseases stem from the expansion of CAG repeats in particular genes with the encoded proteins having corresponding polyglutamine tracts which lead to aggregation and accumulation in the nuclei and cytoplasm of neurons. Aggregated amino-terminal fragments of mutant huntingtin are toxic to neuronal cells and are thought to mediate neurodegeneration. Examples include Huntington's disease (HD), which is characterized by selective neuronal cell death primarily in the cortex and striatum. CAG expansions have also been found in at least seven other inherited neurodegenerative disorders, including for example spinal and bulbar muscular atrophy (SBMA), Kennedy's disease, some forms of amyotrophic lateral sclerosis (ALS), dentatorubral pallidoluysian atrophy (DRPLA) and spinocerebellar ataxia (SCA) types 1, 2, 3, 6 and 7.

Gene therapies according to the invention finds particular application in the treatment of any neoplasia, including proliferative disorders, benign, pre-cancerous and malignant neoplasia, hyperlasia, metaplasia and dysplasia. The invention therefore finds application in the treatment of proliferative disorders which include, but are not limited to cancer, cancer metastasis, smooth muscle cell proliferation, systemic sclerosis, cirrhosis of the liver, adult respiratory distress syndrome, idiopathic cardiomyopathy, lupus erythematosus, retinopathy (e.g. diabetic retinopathy), cardiac hyperplasia, benign prostatic hyperplasia, ovarian cysts, pulmonary fibrosis, endometriosis, fibromatosis, haematomas, lymphangiomatosis, sarcoidosis and desmoid tumours. Neoplasia involving smooth muscle cell proliferation include hyperproliferation of cells in the vasculature (e.g., intimal smooth muscle cell hyperplasia, restenosis and vascular occlusion, including in particular stenosis following biologically- or mechanically mediated vascular injury, such as angioplasty). Moreover, intimal smooth muscle cell hyperplasia can include hyperplasia in smooth muscle other than the vasculature (e.g., blockage of the bile duct, bronchial airways and in the kidneys of patients with renal interstitial fibrosis). Non-cancerous proliferative disorders also include hyperproliferation of cells in the skin such as psoriasis and its varied clinical forms, Reiter's syndrome, pityriasis rubra pilaris and hyperproliferative variants of disorders of keratinization (including actinic keratosis, senile keratosis and scleroderma). Particularly preferred is the treatment of malignant neoplasia (cancer).

The term “pharmaceutically acceptable excipient” refers to a diluent, adjuvant, excipient, or vehicle with which the polyplex is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

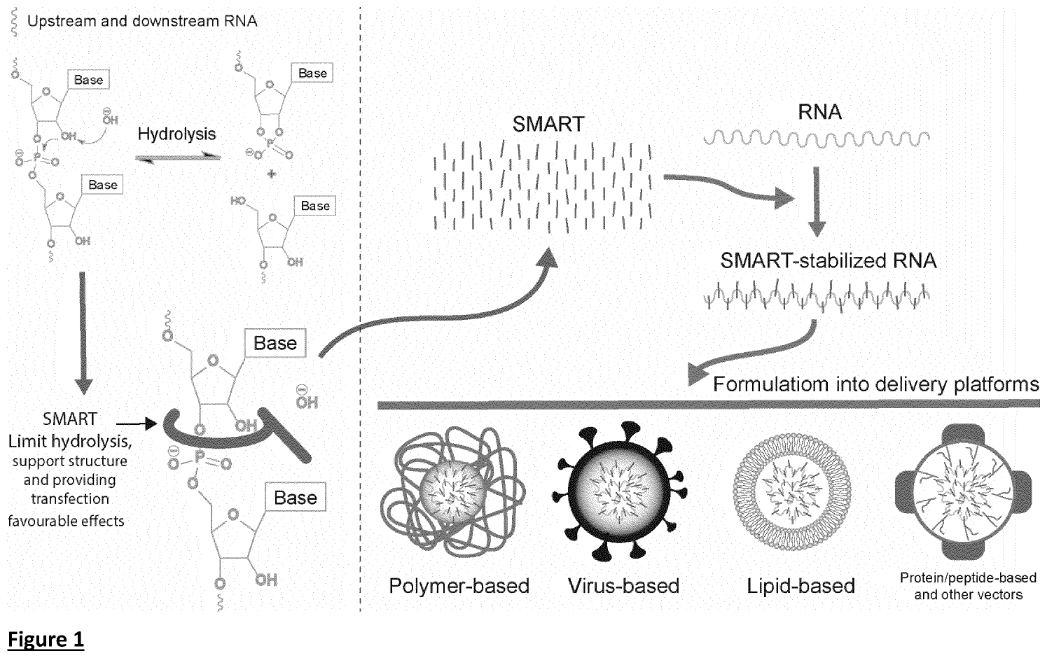

The inventors found that unexpectedly, the admixture of a specific group of small molecules into RNA delivery vectors dramatically increases transfection efficacy, even one-two order of magnitudes, and significantly improves their stability. Furthermore, these unexpected small molecule admixtures were found to contribute to the stability and efficacy of the delivery of other nucleic acids and gene editing systems. Thus, the inventors named it Small Molecules Admixture for Readily Transfection (SMART), which was also found generalisable for polymer-based, lipid-based and other delivery platforms in the follow-up exploration (FIG. 1).

An example library of SMART is provided above. The positive-charged SMART family contains at least one atom/group that carry positive charges in aqueous solutions via protonation, quaternisation, or similar reactions/processes for nucleic acids and some peptide-based structures. The phosphodiester bonds of RNA/DNA tend to be negatively charged in formulation buffers. Thus, SMART can bind to RNA/DNA at phosphodiester bond and block auto-hydrolysis or attack from RNase/DNase. More importantly, SMART shall not carry structure/groups or too many charged atoms/groups that hinder optimal interaction with RNA/DNA/proteins' secondary structures and subsequent RNA/DNA/protein encapsulations within delivery vectors. On the other hand, the negative-charged SMART family can bind to positively charged peptide-based structures, enhancing stability and transfection efficacy in similar manners.

A commercial lipid-based mRNA transfection reagent, Lipofectamine MessengerMAX (Lipo MM), a commercial polymer-based mRNA transfection reagent, jetMESSENGER (jetM), a commercial polymer-based transfection reagent carrying high efficacy for DNA but insufficient for mRNA, Xfect, and a lab-synthesized hyperbranched poly beta-amino ester polymer more efficient for DNA (HPAE-control) from patent WO2021058491A1 (Scheme 3) have been selected as delivery platforms to test SMART. The luciferase mRNA (Luc mRNA) was mixed with SMART. A Positive Charge Group/Phosphodiester Bond ratio at ten was used as an example, and the ratio may vary from 500 to 0.1. Then the SMART-mRNA was formulated with the lipid- or polymer-based delivery platforms following standard commercial and patented protocols accordingly.

The HPAE used is the backbone consists of B4, S5 and PTTA, endcapped with 122, as described in WO2021058491A1

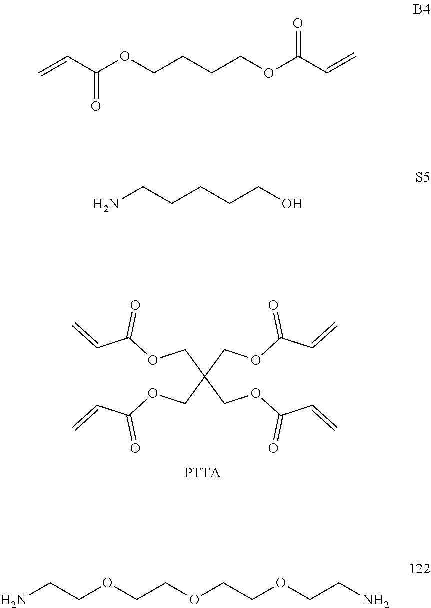

To make the HPAE: backbone monomers are first weighted and dissolved/mixed in DMSO to desired concentrations and put in an oil bath at 90° C. for polymerization. When the desired molecular weight is reached, dilute the reaction mixture with DMSO and add endcap monomer to the desired concentration for end-capping reaction at room temperature. After completing the end-capping, the polymer DMSO solution is purified with diethyl ether wash and collected and dried under a vacuum. The final product is then dissolved in DMSO to 100 mg/ml as a stock solution.

mRNA-HPAE-Control Polyplex, Stabilized by SMART (HPAE-SMART-Stable):
1. The SMART can be introduced into the polyplex at least in 5 ways: 1) add in mixture buffer in advance; 2) add in mRNA buffer solution; 3) add in polymer buffer solution; 4) add in mRNA stock solution; 5) add in polymer stock solution.
2. After the addition of SMART, operate the polyplex mixture in the usual way or any optimized way, for example:
   2.1 Add SMART in mixture buffer in advance;
   2.2 Dilute mRNA in the buffer containing SMART;
   2.3 Dilute polymer (HPAE-control) in the buffer in a separate tube;
   2.4 Add diluted polymer solution into diluted mRNA solution with SMART binding on mRNA;
   2.5 Incubate for the required time (i.e., 1-10 min) to form HPAE-SMART-stable polyplex;
   2.6 Use for transfection freshly or lyophilization for storage as drug products.

### Cell Transfection Efficiency:

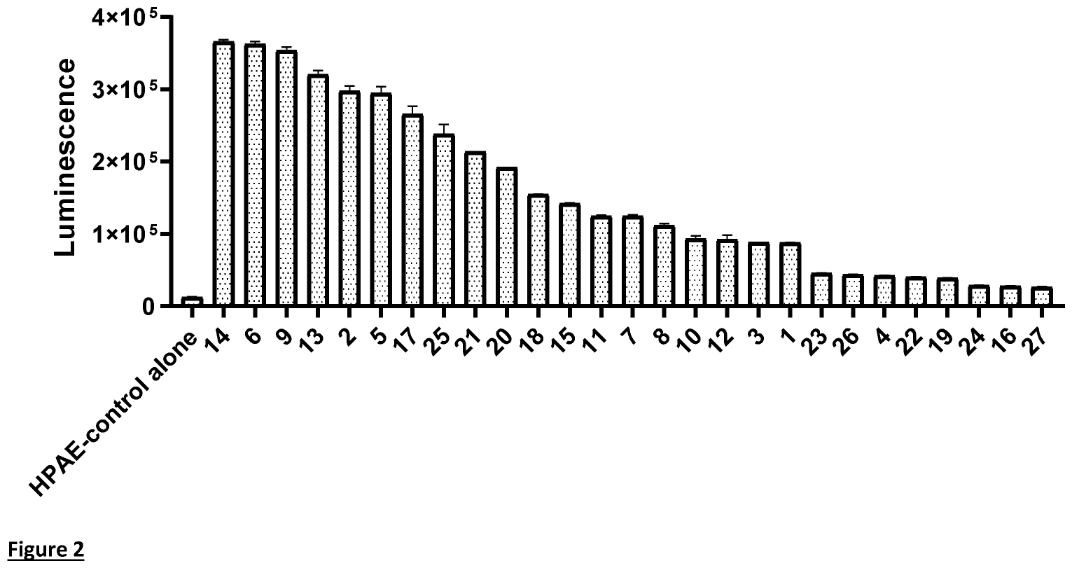

The luminescence intensities represent luciferase protein expression after 48-hour post-transfection of Human Embryonic Kidney cells (HEK293). The majority from the example library of SMART showed enhanced efficacy when they were co-formulated into the delivery systems. The HPAE-control was screened for DNA delivery and found to be insufficient to deliver mRNA alone. However, with the optimal SMART from the library, the mRNA delivery efficacy was enhanced by more than one order of magnitude (FIG. 2).

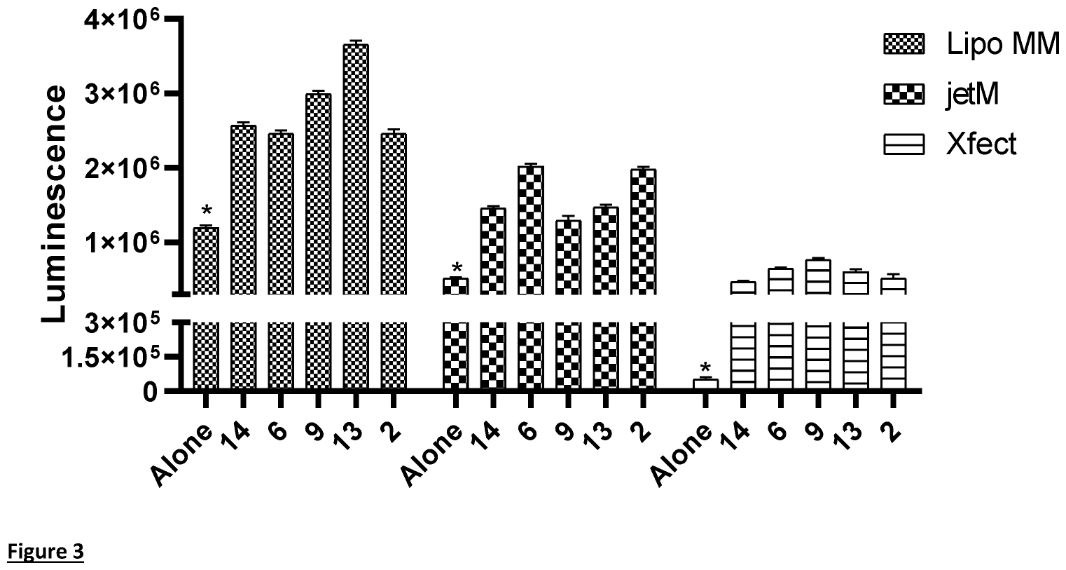

Then the optimal five SMART candidates were selected to test with Lipo MM, jetM and Xfect, which presented significant enhancement of efficacy within all groups (FIG. 3). Especially for Xfect, the nearly two orders of magnitude enhancement are considered due to the specifically enhanced stability of RNA. The Lipo MM and jetM were designed and screened for mRNA delivery and carry some inherent RNA stabilising ability. However, the potential RNA-stabilizing structures within lipids or polymers may not cover all phosphodiester bonds of RNA. Therefore, SMART also significantly increased their mRNA delivery efficacy on account of their advantages as small molecules.

### Cell Transfection Viability:

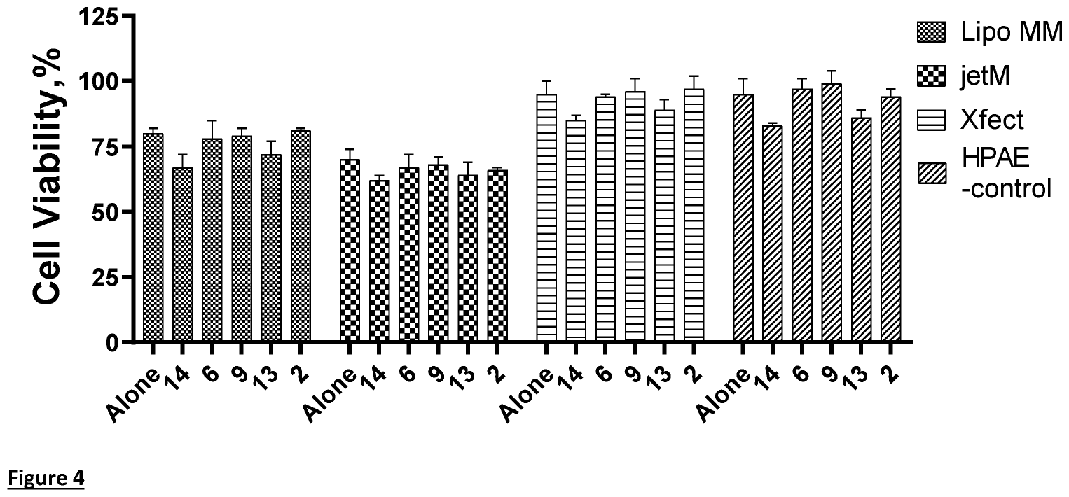

Transfected HEK293 with SMART at 0.1 μg mRNA for luciferase expression showed high viability, comparable to control and untreated ones (set as 100% viability) (FIG. 4).

mRNA Entrapment, Size and Stability:

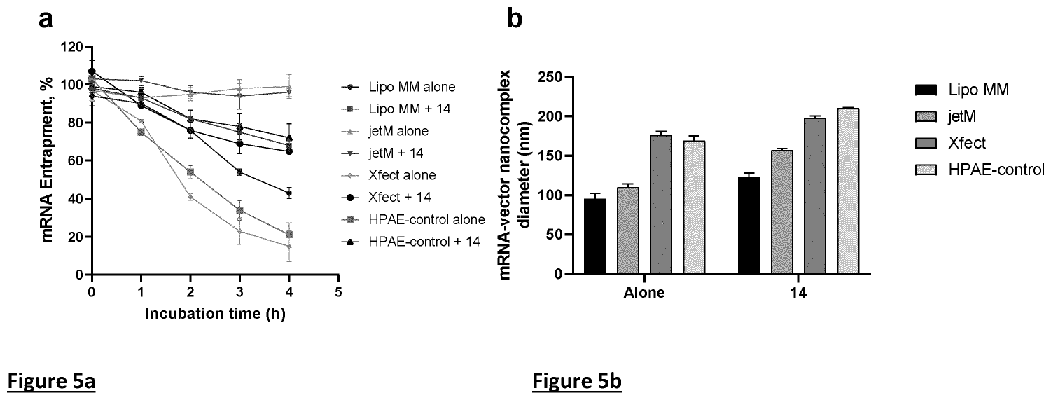

SMART candidate 14 then was selected to test its effect on the mRNA entrapment, mRNA-vector complex size and stability. The co-formulation with SMART candidate 14 increased the mRNA entrapment and stability of both lipid-based (Lipo MM) and degradable polymer-based (Xfect and HPAE-control) vectors (FIG. 5a) but also slightly increased the size of mRNA-vector nanocomplexes for all groups (FIG. 5b).

### Thermal Stability of SMART-Formulated Fresh and Lyophilised Polyplex:

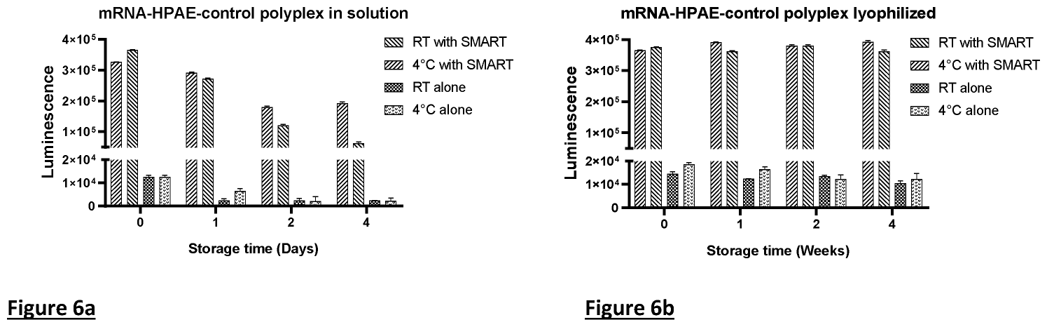

SMART candidate 14 and HPAE-control were selected as an example to investigate further and demonstrate the thermal stability of mRNA enhanced by SMART. HPAE-control is degradable in water, so the fresh mRNA-HPAE control polyplex lost efficacy quickly at room temperature and 4 degrees Celsius. However, the existence of SMART decreased the loss of efficacy of fresh polyplex in water. Furthermore, after lyophilisation, with limited moisture remaining, SMART-formulated polyplex remained nearly 100% efficacy for long terms when stored at room temperature and 4 degrees Celsius (FIG. 6).

By the admixture and co-formulation, SMART can (1) reduce storage and transportation cost and prolong storage time of gene-related formulation; (2) preserve the functions of active drug substances during administration route to enhance the treatment efficacy; (3) help endosome release to increase the efficiency for vectors that cannot penetrate cell membrane directly; (4) potentially decrease the undesired inflammatory reactions to both broken and intact active drug substances. Once in the cell cytoplasm, the SMART co-formulated delivery vectors break down. SMART is then released and metabolised.

Using SMART, current gene delivery techniques can be boosted for broader and more robust medical applications, including treatment of genetic diseases, inflammatory diseases, cancer and vaccination. For the delivery system to be co-formulated with SMART, the active part can be any natural or modified DNA, RNA, gene editing associated systems or other bioactive molecules sensitive to hydrolysis, enzymes and other damaging factors, and the inactive part, delivery vectors, can be any vectors that deliver active substances.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCES

1. Lallana, E. et al. Chitosan/Hyaluronic Acid Nanoparticles: Rational Design Revisited for RNA Delivery. Mol. Pharm. 14, 2422-2436 (2017).
2. Siewert, C. et al. Investigation of charge ratio variation in mRNA-DEAE-dextran polyplex delivery systems. Biomaterials 192, 612-620 (2019).
3. Miyazaki, T. et al. Polymeric Nanocarriers with Controlled Chain Flexibility Boost mRNA Delivery In Vivo through Enhanced Structural Fastening. Adv. Healthc. Mater. 9, (2020).
4. Ke, X. et al. Surface-Functionalized PEGylated Nanoparticles Deliver Messenger RNA to Pulmonary Immune Cells. ACS Appl. Mater. Interfaces 12, 35835-35844 (2020).
5. Kauffman, K. J., Webber, M. J. & Anderson, D. G. Materials for non-viral intracellular delivery of messenger RNA therapeutics. J. Control. Release 240, 227-234 (2016).
6. Love, K. T. et al. Lipid-like materials for low-dose, in vivo gene silencing. Proc. Natl. Acad. Sci. U.S.A. 107, 1864-1869 (2010).
7. Hajj, K. A. et al. A Potent Branched-Tail Lipid Nanoparticle Enables Multiplexed mRNA Delivery and Gene Editing in Vivo. Nano Lett. 20, 5167-5175 (2020).
8. Fenton, O. S. et al. Bioinspired Alkenyl Amino Alcohol Ionizable Lipid Materials for Highly Potent in Vivo mRNA Delivery. Adv. Mater. 28, 2939-2943 (2016).
9. Li, B. et al. An Orthogonal Array Optimization of Lipid-like Nanoparticles for mRNA Delivery in Vivo. Nano Lett. 15, 8099-8107 (2015).
10. Zhou, K. et al. Modular degradable dendrimers enable small RNAs to extend survival in an aggressive liver cancer model. Proc. Natl. Acad. Sci. U.S.A. 113, 520-525 (2016).
11. Sabnis, S. et al. A Novel Amino Lipid Series for mRNA Delivery: Improved Endosomal Escape and Sustained Pharmacology and Safety in Non-human Primates. Mol. Ther. 26, 1509-1519 (2018).
12. Chaudhary, N., Weissman, D. & Whitehead, K. A. mRNA vaccines for infectious diseases: principles, delivery and clinical translation. Nat. Rev. Drug Discov. 0123456789, (2021).
13. van den Brand, D. et al. Peptide-mediated delivery of therapeutic mRNA in ovarian cancer. Eur. J. Pharm. Biopharm. 141, 180-190 (2019).
14. Armbruster, N., Jasny, E. & Petsch, B. Advances in RNA Vaccines for Preventive Indications: A Case Study of a Vaccine against Rabies. Vaccines 2019, Vol. 7, Page 132 7, 132 (2019).
15. Segel, M. et al. Mammalian retrovirus-like protein PEG10 packages its own mRNA and can be pseudotyped for mRNA delivery. Science (80-.). 373, 882-889 (2021).
16. Brito, L. A. et al. A cationic nanoemulsion for the delivery of next-generation RNA vaccines. Mol. Ther. 22, 2118-2129 (2014).
17. Tsai, T. F. Fluad®-mf59®-adjuvanted influenza vaccine in older adults. Infect. Chemother. 45, 159-174 (2013).

1. A nanoparticulate composition comprising (a) a core comprising a therapeutically active nucleic acid packaged within (b) a non-viral polymer, lipid or protein-based drug delivery vehicle, characterized in that the core comprises a low molecular weight stabilizing agent comprising at least one atom or group that is charged in aqueous solution, wherein when the therapeutic agent is negatively charged the charged atom or group is positively charged in aqueous solution and when the therapeutic agent is positively charged the charged atom or group is negatively charged in aqueous solution in which the low molecular weight stabilizing agent has a chemical formula R₁-L₁-R₂;
in which:
R₁ is selected from a primary, secondary or tertiary amino or hydroxyl group;
L₁ is an alkyl group which is straight or branched and optionally substituted
R₃ is selected from a primary, secondary or tertiary amine or a hydroxyl group or is absent.

2. The nanoparticulate composition according to claim 1 formed by
(a) admixing the therapeutically active nucleic acid with the low molecular weight stabilizing agent to form a stabilized therapeutically active agent;
(b) admixing the stabilized therapeutically active agent with the non-viral polymer, lipid or protein-based drug delivery vehicle to form the nanoparticulate composition; and
(c) optionally, lyophilizing the nanoparticulate composition.

3. The nanoparticulate composition according to claim 1, in which the low molecular weight stabilizing agent has a molecular weight of less than 5000 Da.

4. The nanoparticulate composition according to claim 1, in which the low molecular weight stabilizing agent has a molecular weight of less than 2000 Da.

5. The nanoparticulate composition according to claim 1, in which the low molecular weight stabilizing agent has a molecular weight of less than 500 Da.

6. The nanoparticulate composition according to claim 1, in which:
R₁ is NH₂;
L₁ is selected from a straight or branched amine or alkyl group having 2 to 8 carbon atoms; and
R₃ is selected from a primary, secondary or tertiary amine.

7. The nanoparticulate composition according to claim 1, in which R₃ is selected from the groups consisting of: NH₂; N(CH₃)₂; and a heterocyclic group in which at least one ring atom is nitrogen.

8. The nanoparticulate composition according to claim 7, in which the heterocyclic group has a chemical structure selected from: 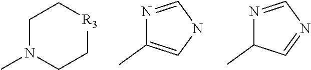
in which R3 is CH or comprises a heteroatom such as N or O.

9. The nanoparticulate composition according to claim 1, in which the low molecular weight stabilizing agent is selected from:

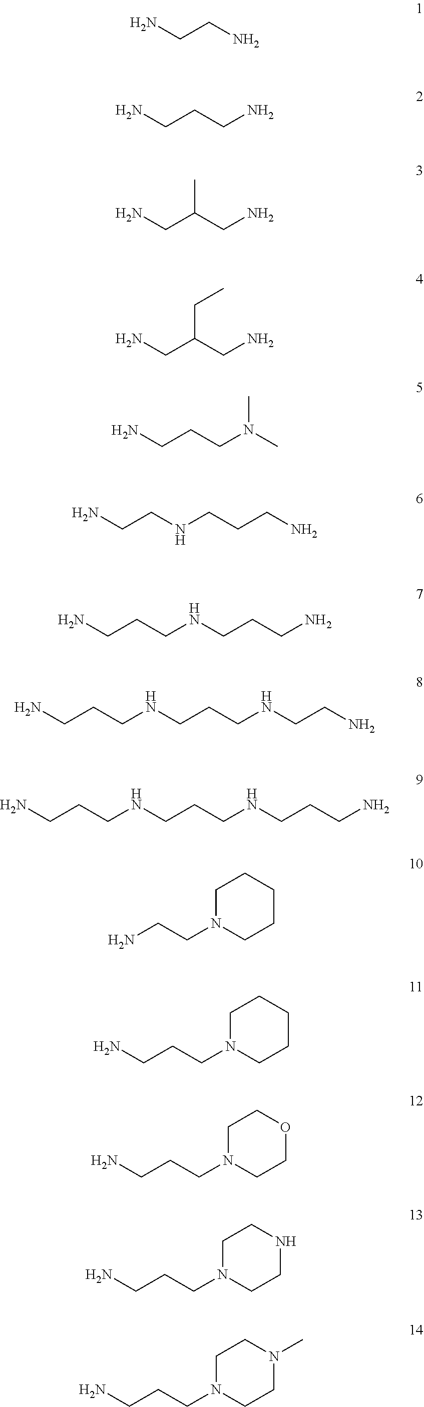

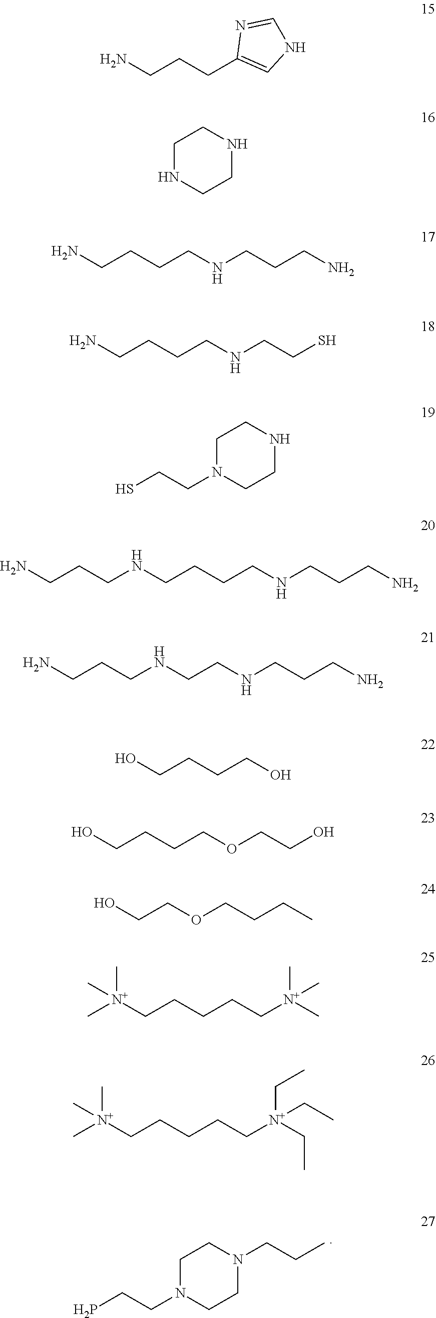

10. The nanoparticulate composition according to claim 1, in which the therapeutically active nucleic acid is selected from long RNA and DNA.

11. The nanoparticulate composition according to claim 1, in which the therapeutically active nucleic acid is long RNA.

12. The nanoparticulate composition according to claim 1, in which the drug delivery vehicle is a cationic polymer-based drug delivery vehicle.

13. The nanoparticulate composition according to claim 1, in a lyophilized form.

14.-16. (canceled)
